(19) United States
(12) Patent Application Publication
(10) Pub. No.: US 2015/0336926 A1
(43) Pub. Date: Nov. 26, 2015

(54) FLUORINATION OF ARYL COMPOUNDS

(71) Applicant: THE REGENTS OF THE UNIVERSITY OF CALIFORNIA, Oakland, CA (US)

(72) Inventors: John HARTWIG, Berkeley, CA (US); Patrick FIER, Berkeley, CA (US)

(21) Appl. No.: 14/758,906

(22) PCT Filed: Dec. 23, 2013
(86) PCT No.: PCT/US13/77602

§ 371 Date: Jul. 1, 2015

Related U.S. Application Data
(60) Provisional application No. 61/748,116, filed on Jan. 1, 2013.

(51) Int. Cl. C07D 333/76; C07C 41/22; C07C 231/12; C07C 67/307; C07B 39/00; C07C 269/06; C07C 253/30; C07D 209/08; C07J 1/00; C07D 209/88; C07C 17/361; C07C 45/63

(52) U.S. Cl. CPC C07D 333/76; C07C 17/361; C07C 41/22; C07C 231/12; C07C 67/307; C07C 45/63; C07C 269/06; C07C 253/30; C07D 209/08; C07J 1/00; C07D 209/88; C07B 39/00

(57) ABSTRACT

The invention provides compositions and methods of using the compositions in fluorinating aryl precursors containing a leaving group replaceable by a fluorine atom. The compositions include a metal ion source, a electrophilic fluorine source, a base, and a compound, which is an aryl precursor of the aryl fluoride, and which has a leaving group replaceable by the fluorine atom. Exemplary methods of the invention make use of such compositions and methods to prepare an aryl fluoride compound. In an exemplary embodiment, the electrophilic fluorine source is a source of ¹⁸F.

### CROSS-REFERENCE TO RELATED APPLICATIONS

This application claims under 35 USC 119(e) the benefit of U.S. Provisional Application No. 61/748,116, filed Jan. 1, 2013, which is incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with Government support under Grant No. GM-55382 awarded by the National Institutes of Health. The Government has certain rights in this invention.

### BACKGROUND OF THE INVENTION

A wide range of materials and biologically active molecules contain fluoroarenes. The presence of fluorine atoms in these arenes often affects reactivity, solubility, and stability of the molecule. In medicinal chemistry, a fluorine atom is used to block metabolic degradation and, thereby, to improve the efficacy of lead compounds. In addition, fluorinated compounds enriched in ¹⁸F are used as PET-imaging agents in medicine. However, methods to synthesize aryl fluorides under mild reaction conditions are limited.

To overcome the limitations of classical methods for the synthesis of aryl fluorides by the Halex [Adams, D. J.; Clark, J. H. Chem. Soc. Rev. 1999, 28, 225.] or Balz-Schieman reactions (Scheme 1), [Olah, G. A.; Welch, J. T.; Vankar, Y. D.; Nojima, M.; Kerekes, I.; Olah, J. A. J. Org. Chem. 1979, 44, 3872.] modern methods based on transition metal complexes have been sought (Scheme 2). Aryl triflates react with CsF in the presence of a palladium catalyst to form aryl fluorides, but isomeric products were obtained in many cases. [Watson, D. A.; Su, M. J.; Teverovskiy, G.; Zhang, Y.; Garcia-Fortanet, J.; Kinzel, T.; Buchwald, S. L. Science 2009, 325, 1661.] Arylstannanes, [a) Furuya, T.; Strom, A. E.; Ritter, T. J. Am. Chem. Soc. 2009, 131, 1662; b) Tang, P. P.; Furuya, T.; Ritter, T. J. Am. Chem. Soc. 2010, 132, 12150.] arylsilver, [Furuya, T.; Ritter, T. Org. Lett. 2009, 11, 2860.] arylpalladium, [Furuya, T.; Kaiser, H. M.; Ritter, T. Angew. Chem. Int. Ed. 2008, 47, 5993.] and arylnickel [Lee, E.; Hooker, M. H.; Ritter, T. J. Am. Chem. Soc. 2012, 134, 17456.] complexes have been reported to form aryl fluorides, but the stannanes are toxic, and the silver, palladium, and nickel complexes must be isolated after synthesis from arylboronic acids (Ag, Pd) or aryl bromides (Ni). More recently, Ritter and coworkers reported the direct conversion of phenols to aryl fluorides with a difluoroimidazoline reagent, [Tang, P. P.; Wang, W. K.; Ritter, T. J. Am. Chem. Soc. 2011, 133, 11482.] and we disclosed the conversion of aryl iodides to aryl fluorides with (^{t}BuCN)₂CuOTf and AgF. [Fier, P. S.; Hartwig, J. F. J. Am. Chem. Soc. 2012, 134, 10795.]

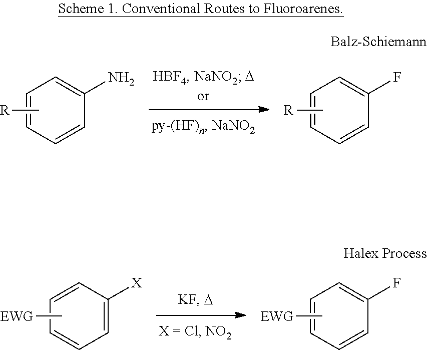

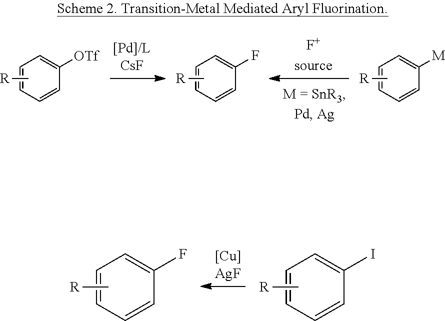

Arylboron reagents are valuable alternative sources of aryl groups for the synthesis of aryl fluorides because they are readily available, non-toxic, shelf-stable, and often react under mild-conditions with good functional group tolerance. Moreover, they can be prepared by methods, such as C—H bond functionalization, that complement those used to form aryl iodides and phenols. Finally, reactions of arylboronate esters can occur with reactivity that is orthogonal to that of aryl iodides. However, no direct conversion of arylboron reagents to aryl fluorides has been reported.

Accordingly, a reaction that directly fluorinates an aryl precursor to form the corresponding aryl fluoride at low to modest temperatures (e.g., <300° C.) would represent a significant advance in the art of aryl fluorination and the provision of aryl fluorides. Further, such a reaction that does not require the presence of electron withdrawing substituents on the aryl nucleus would also be of value. Surprisingly, the present invention provides such a reaction and compositions of use in carrying out this reaction.

### BRIEF SUMMARY OF THE INVENTION

The present invention provides compositions and methods for fluorinating functionally diverse aryl precursor compounds with a simple metal reagent and electrophilic fluorine source. In various embodiments, the metal is complexed with a ligand. The reaction occurs at low to modest temperatures, allowing the presence of diverse substituents on the aryl nucleus. Furthermore, the presence of electron withdrawing substituents on the aryl ring is not required.

In general terms, the invention provides a method of aryl fluorination and compositions of use therein:

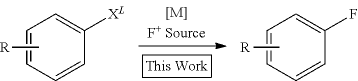

in which X^{L} is a leaving group, M is a metal and the F⁺ source is an electrophilic fluorine source. In an exemplary embodiment, [M] is a liganded copper ion.

The invention provides an operationally simple fluorination of aryl precursor compounds with readily available reagents. This reaction tolerates a range of functional groups other than the leaving group, e.g., ester, ketone, aldehyde, amide, nitrile, and halogen functional groups and occurs with heterocyclic systems. Moreover, it occurs in moderate to good yield with sterically hindered aryl precursor compounds. Also provided are compositions and methods for the synthesis of ¹⁸F labeled compounds, which, in an exemplary embodiment, are of use in PET imaging.

Thus, in an exemplary embodiment, there is provided a reaction mixture for fluorinating an aryl precursor compound having a leaving group. The reaction mixture includes: (i) the aryl precursor compound, which is optionally further substituted at one or more positions other than the position occupied by the leaving group; (ii) an electrophilic fluorine source; (iii) a metal source, wherein the metal source mediates the fluorinating of the aryl precursor compound at the position of the leaving group with fluorine derived from the electrophilic fluorine source; and (iv) a base.

Also provided is a method of fluorinating an aryl precursor compound having a leaving group, which is replaceable by fluorine from an electrophilic fluorine source. The method includes forming a reaction mixture according to the invention and incubating the reaction mixture under conditions appropriate to form the fluoroaryl compound.

Other exemplary objects, advantages and aspects of the invention are set forth in the detailed description that follows.

### DETAILED DESCRIPTION OF THE INVENTION

### I. Introduction

The ability to selectively fluorinate an aryl substrate has broad application, especially in the agricultural, pharmaceutical, and polymer industries. As described herein, the present invention relates to compositions and methods for transforming an aryl substrate to the corresponding fluoro compound. The compositions and methods of the invention utilize simple, readily available substrates and reaction mixtures and, thus, have wide applicability.

In various embodiments, the present invention provides a one-step procedure for the fluorination of aryl substrates that occurs with readily available and non-hazardous reagents. This reaction tolerates a wide range of substituents, e.g., ester, ketone, aldehyde, amide, nitrile, and halogen functionalities, and occurs in moderate to good yield even with sterically hindered substrates. The simplicity and generality of this method makes it attractive for the introduction of fluorine into functionally diverse aryl compounds.

In various embodiments, there is provided a reaction mixture for fluorinating an aryl precursor compound having a leaving group, said reaction mixture comprising: (i) the aryl precursor compound, which is optionally further substituted at one or more positions other than the position occupied by the leaving group; (ii) an electrophilic fluorine source; (iii) a metal source; and (iv) a base. The metal ion source mediates the fluorinating of the aryl substrate at the position of the leaving group with fluorine derived from the electrophilic fluorine source.

Also provided is a method of utilizing such a reaction mixture to prepare an aryl fluoride compound. In general terms, the method includes incubating the reaction mixture under conditions sufficient to form the aryl fluoride.

Before the invention is described in greater detail, it is to be understood that the invention is not limited to particular embodiments described herein as such embodiments may vary. It is also to be understood that the terminology used herein is for the purpose of describing particular embodiments only, and the terminology is not intended to be limiting. The scope of the invention will be limited only by the appended claims. Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

In describing the present invention, the following terms will be employed, and are defined as indicated below.

### II. DEFINITIONS

Where substituent groups are specified by their conventional chemical formulae, written from left to right, the structures optionally also encompass the chemically identical substituents, which would result from writing the structure from right to left, e.g., —CH₂O— is intended to also optionally recite —OCH₂—.

The term “alkyl,” by itself or as part of another substituent, means, unless otherwise stated, a straight or branched chain, or cyclic hydrocarbon radical, or combination thereof, which may be fully saturated, mono- or polyunsaturated and can include di-, tri- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to optionally include those derivatives of alkyl defined in more detail below, such as “heteroalkyl.” Alkyl groups that are limited to hydrocarbon groups are termed “homoalkyl”. Exemplary alkyl groups include the monounsaturated C₉₋₁₀, oleoyl chain or the diunsaturated C_{9-10, 12-13} linoeyl chain.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The terms “aryloxy” and “heteroaryloxy” are used in their conventional sense, and refer to those aryl or heteroaryl groups attached to the remainder of the molecule via an oxygen atom.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Further exemplary cycloalkyl groups include steroids, e.g., cholesterol and its derivatives. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxyl)propyl, and the like).

Each of the above terms (e.g., “alkyl,” “heteroalkyl,” “aryl” and “heteroaryl”) are meant to optionally include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: H, substituted or unsubstituted aryl, substituted or unsubstituted heteroaryl, substituted or unsubstituted heterocycloalkyl, —OR′, ═O, ═NR′, ═N—OR′, —NR′R″, —SR′, halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like). These terms encompass groups considered exemplary “alkyl group substituents”, which are components of exemplary “substituted alkyl” and “substituted heteroalkyl” moieties.

Similar to the substituents described for the alkyl radical, substituents for the aryl and heteroaryl groups are generically referred to as “aryl group substituents.” The substituents are selected from, for example: H, substituted or unsubstituted alkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl. These terms encompass groups considered exemplary “aryl group substituents”, which are components of exemplary “substituted aryl” and “substituted heteroaryl” moieties.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non-aromatic rings. Examples of “fused ring systems” are naphthalenes, indoles, quinolines, chromenes and the like.

As used herein, the term “heteroatom” includes oxygen (O), nitrogen (N), sulfur (S) and silicon (Si) and boron (B).

The symbol “R” is a general abbreviation that represents a substituent group that is selected from H, substituted or unsubstituted alkyl, substituted or unsubstituted heteroalkyl, substituted or unsubstituted aryl, substituted or unsubstituted heteroaryl, and substituted or unsubstituted heterocycloalkyl groups.

The terms “substrate” and “precursor” are used interchangeably and refer to compound with a leaving group substitutable by a fluorine synthon in a method and composition of the invention. An exemplary substrate or precursor is an iodo-substituted aryl compound, which can react under the conditions of the invention, to yield at least one product having a fluoro moiety.

The compounds disclosed herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Useful leaving groups include, but are not limited to, halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates). Exemplary leaving groups include a halogen, B(OR³⁶)(OR³⁷), OC(O)R³⁶, OP(O)R³⁶R³⁷, OS(O)R³⁶, OSO₂R³⁶, SR³⁶, (R³⁶)₃P⁺, (R³⁶)₂S⁺, P(O)N(R³⁶)₂(R³⁶)₂, P(O)R³⁸R³⁶R³⁹R³⁶ in which each R³⁶ and R³⁷ is independently selected from H, substituted or unsubstituted alkyl, substituted or unsubstituted aryl, substituted or unsubstituted heteroaryl and substituted or unsubstituted heterocycloalkyl. R³⁸ and R³⁹ are each either S or O. When the leaving group is a boric acid ester, it is optionally a cyclic boronic acid ester.

The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, Compendium OF ORGANIC SYNTHETIC METHODS, John Wiley and Sons, 1980).

The term “ligand” has the meaning ordinarily ascribed to it in the art. Exemplary ligands include at least one donor atom capable of binding to Cr, Mn, Fe, Co, Cu, Ni, Pd, Rh, Ag or Pt. In an exemplary embodiment, the ligand includes at least one donor atom capable of binding to copper (e.g., Cu(0), Cu(I) or Cu(II). Ligands can include sterically bulky species, such as substituted or unsubstituted aryl, substituted or unsubstituted heteroaryl and substituted or unsubstituted fused ring systems, secondary and tertiary alkyl groups and the like. Exemplary ligands include, without limitation, nitrogen-containing ligands and oxygen-containing ligands (e.g., nitriles, amines, aminoalcohols, amino acids, phenols), and phosphorus-containing ligands (e.g., phosphines and phosphites). An exemplary ligand is a substituted or unsubstituted alkyl nitrile or a substituted or unsubstituted aryl nitrile.

The term “salt(s)” includes salts of the compounds prepared by the neutralization of acids or bases, depending on the particular ligands or substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium salt, or a similar salt. Examples of acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids, and the like, as well as the salts derived from relatively nontoxic organic acids like acetic, propionic, isobutyric, butyric, maleic, malic, malonic, benzoic, succinic, suberic, fumaric, lactic, mandelic, phthalic, benzenesulfonic, p-tolylsulfonic, citric, tartaric, methanesulfonic, and the like. Certain specific compounds of the present invention contain both basic and acidic functionalities that allow the compounds to be converted into either base or acid addition salts. Hydrates of the salts are also included.

The symbol [symbol] , displayed perpendicular to a bond, indicates the point at which the displayed moiety is attached to the remainder of the molecule.

A “boronic acid derivative” refers, inter alia, to boronate esters (e.g., arylboronate esters). Exemplary boronic acid derivatives include at least one, aryl group, one amino, one alkoxy group or a combination thereof.

As used herein, an “electrophilic fluorine source” includes, without limitation, pyridinium fluorides, ammonium fluorides and fluorinated imides. Specific examples include F-TEDA-BF₄, [Cl₂pyF]OTf; [pyF]OTf; [Me₃pyF]BF₄; [Me₃pyF]OTf; and [Me₃pyF]PF₆, and NFSI.

Below are examples of specific embodiments for carrying out the present invention. The examples are offered for illustrative purposes only, and are not intended to limit the scope of the present invention in any way.

In some embodiments, the definition of terms used herein is according to IUPAC.

### III. The Compositions

In an exemplary embodiment, the invention provides a reaction mixture that includes an aryl precursor compound with a leaving group, the metal source (liganded or unliganded), the electrophilic fluorine source, and the base. In various embodiments, the reaction mixture also contains an appropriate solvent for at least one of the components of the reaction mixture.

IIIa. Aryl Precursor Compound

The aryl precursor compound includes at least one leaving group. Useful leaving groups are conveniently selected from any such group that can be substituted by a fluorine atom or fluorine synthon using a reaction mixture of the invention in a method of the invention. In various embodiments, the leaving groups are selected from a boronic acid moiety, a boronic acid derivative (such as a boronate ester), and a boronic acid surrogate (such as trifluoroborate). Other appropriate leaving groups will be apparent to those of skill in the art.

The reaction mixture functions to transform aryl substrates of a broad range of structures to fluoroaryl compounds. For example, in addition to the leaving group, the precursor is optionally further substituted with an ester, ketone, aldehyde, amide, nitrile, halogen, heterocycle or a combination thereof. The metal mediates the transfer of the fluorine from the electrophilic fluorine source to the position of the aryl ring occupied by the leaving group.

In an exemplary embodiment, the aryl precursor compound has the formula:

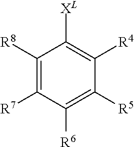

wherein R⁴, R⁵, R⁶, R⁷, and R⁸ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R⁴, R⁵, R⁶, R⁷ and R⁸, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl. X^{L} is a leaving group.

The symbols R⁹ and R¹⁰ represent members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl.

In some embodiments, the aryl precursor compound was synthesized in situ. In some embodiments, the aryl precursor compound was synthesized in situ from an arene or an aryl halide.

### Leaving Group

In an exemplary embodiment, the leaving group is not SnR₃, Pd, Ag, or Ni. In an exemplary embodiment, the leaving group does not include Sn, Pd, Ag, or Ni.

In an exemplary embodiment, the leaving group is a boron leaving group. As used herein, the term “boron leaving group” refers to a boron-containing leaving group that is attached to the aryl ring of the aryl precursor compound through the boron (such as —B(OH)₂).

In an exemplary embodiment, the leaving group is a member selected from a boronic acid moiety, a boronic acid derivative and a boronic acid surrogate (such as trifluoroborate). In an exemplary embodiment, the leaving group is a boronate ester moiety. In an exemplary embodiment, the leaving group is a member selected from —B(OH)₂; —BF₃K;

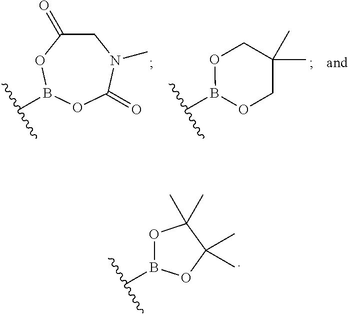

IIIb. Electrophilic Fluorine Source

A variety of electrophilic fluorine sources are known in the art and readily available. Exemplary electrophilic fluorine sources are fluoroammonium salts, fluoropyridinium salts, fluoroaminosulfuranes (Et₂NSF₃ (DAST), (Me₂N)₃S(Me)₃SiF₂ (TASF), and difluoroiodobenzene, and xenon difluoride. In an exemplary embodiment, the electrophilic fluorine source is a fluoroammonium salt or a fluoropyridinium salt.

In an exemplary embodiment, the electrophilic fluorine source is a member selected from:

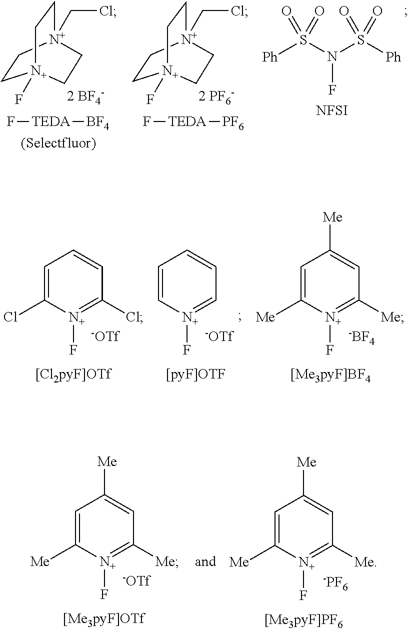

In an exemplary embodiment, the electrophilic fluorine source is a member selected from F-TEDA-BF₄; F-TEDA-PF₆; NFSI; [Me₃pyF]BF₄; [Me₃pyF]OTf; and [Me₃pyF]PF₆.

In an exemplary embodiment, the electrophilic fluorine source comprises:

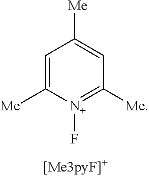

In an exemplary embodiment, the electrophilic fluorine source is:

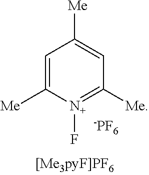

IIIc. Metal Source

The metal source in the reaction mixture can be of any useful formula and form. In various embodiments, the metal is selected from Cr, Mn, Fe, Co, Cu, Ni, Pd, Rh, Ag and Pt. In various embodiments, the metal is Cu(0), Cu(I) or Cu(II). In exemplary embodiments, the metal source is selected from a metal ion and a complex of a metal ion with one or more ligands. In various embodiments, the metal ion is an ion of Cu(0), Cu(I) or Cu(II). In various embodiments, the metal ion is Cu⁺. In an exemplary embodiment, the copper ion source is CuI.

In an exemplary embodiment, the metal ion source has the formula:

(M⁺ⁿ)ₛ(L)ₘ(X^{-t})_{q}

wherein M is the metal ion; L is a ligand, e.g., an organic ligand; X is an anion; m is an integer selected from 0, 1, 2, and 3; and n, s, t and q are integers independently selected from 1, 2 and 3, such that (s×n)=(t×q), or the such that the cationic charge(s) and anionic charge(s) are balanced.

The metal ion is any ion of use to replace a leaving group on an aryl precursor with a fluorine from the electrophilic fluorine source. Exemplary metal ions of use in the present invention include wherein the metal ion is an ion of a member selected from Cr, Mn, Fe, Co, Cu, Ni, Pd, Rh, Ag and Pt. In an exemplary embodiment, the metal ion is Cu⁺.

The ligand is any ligand useful to complex the metal ion and, in an exemplary embodiment, is a substituted or unsubstituted alkyl or substituted or unsubstituted aryl nitrile ligand, RCN. R groups of various substitution patterns are of use in the ligand, reaction mixture and methods of the invention. In an exemplary embodiment, the nitrile is selected for the simplicity of its structure and/or its ready availability. For example, in one embodiment, R is an unsubstituted alkyl, e.g., unsubstituted C₁-C₆ alkyl. In various embodiments, R is selected from an unsubstituted alkyl that does not have an abstractable proton at a position alpha to the cyano moiety. In various embodiments, the nitrile is t-butylnitrile.

The counterion X is selected from organic and inorganic ions to form the corresponding salt. In various embodiments, X is selected from BF₄, PF₆, SbF₆ and OTf, Triflimide (Tf₂N), perchlorate, tetrakis(pentafluorophenyl)borate, tetrakis(3,5-bistrifluoromethylphenyl)borate, Al(OC(CF₃)₃)₄, nonaflate, sulfate, fluorosulfonate, and chlorosulfonate.

In an exemplary embodiment, the metal source is a copper source. In an exemplary embodiment, the copper source is (^{t}BuCN)₂CuOTf.

IIId. Base

In an exemplary embodiment, the base is a fluoride base, an alkoxide base, a phenoxide base, a carbonate base or a phosphate base. In an exemplary embodiment, the base is a fluoride base or an alkoxide base.

In an exemplary embodiment, the fluoride base is a member selected from AgF, KF, NaF, LiF, MgF₂, R₄NF, and CsF. In an exemplary embodiment, the fluoride base is a member selected from AgF, KF, and CsF. In an exemplary embodiment, the fluoride base is AgF.

In various embodiments, the base does not decompose the electrophilic fluorine source.

IIIe. Solvent

The reaction mixture can further include a solvent and this solvent can be any compound or mixture of compounds useful to dissolve at least a portion of one or more component of the reaction mixture. In an exemplary embodiment, the solvent is tetrahydrofuran (THF).

IIIf. Exemplary Compositions/Reaction Mixtures

Any of the combinations of aryl precursor compound, electrophilic fluorine source, metal source, and base are encompassed by this disclosure and specifically provided by the invention.

In some embodiments, the aryl precursor compound is an arylboronate ester and the metal source is (^{t}BuCN)₂CuOTf. In some embodiments, the arylboronate ester is a pinacolate arylboronate ester.

In some embodiments, the aryl precursor compound is an arylboronate ester, the metal source is (^{t}BuCN)₂CuOTf, and the base is a fluoride base. In some embodiments, the fluoride base is AgF.

In some embodiments, the aryl precursor compound is an arylboronate ester, the electrophilic fluorine source comprises [Me₃pyF]⁺, the metal source is (^{t}BuCN)₂CuOTf, and the base is AgF. In some embodiments, the electrophilic fluorine source is [Me₃pyF]PF₆. In some embodiments, the arylboronate ester was synthesized in situ. In some embodiments, the arylboronate ester was synthesized in situ from the corresponding arene or aryl bromide.

In some embodiments, the aryl precursor compound is an aryl boronic acid or a derivative thereof, the electrophilic fluorine source is [Me₃pyF]PF₆, the metal source is (^{t}BuCN)₂CuOTf, and the base is AgF.

Examples of useful aryl precursors, exemplified as their boronate ester analogs, and of their fluoroaryl analogs are set forth in the Examples section. These examples also provide exemplary reactions and yields using ^{t}BuCN-ligated CuOTf. This ligated copper compound can be prepared in multi-gram quantities from Cu₂O, triflic acid and ^{t}BuCN. This complex is stable to oxygen and absorbs moisture from the air only slowly. Thus, this species can be weighed quickly on the benchtop.

As will be appreciated by those of skill in the art, though they generically represent boronate ester compounds, the formulae set forth in the Examples section are equally applicable to precursors substituted with a leaving group which is not a boronate ester moiety.

In various embodiments, the invention provides a reaction mixture in which the molar ratio of the electrophilic fluorine source to the metal (e.g., Cu) is 1 or greater than 1. In various embodiments, the invention provides a reaction mixture in which the aryl precursor compound, the metal source, the electrophilic fluorine source, and the base are present in the reaction mixture in a molar ratio which is about 1:2:3:2. In an exemplary embodiment, the aryl precursor is an aryl boronic acid or a derivative thereof (e.g., a boronate ester, e.g., an arylboronate ester) and the metal source is Cu⁺ in liganded form. In various embodiments, the ligand is t-butyl nitrile.

### IV. The Methods

In various embodiments, the present invention provides methods for converting an aryl precursor compound functionalized with a leaving group to a fluoro aryl compound. In an exemplary embodiment, the method includes: (a) forming a reaction mixture as set forth herein; and (b) incubating the reaction mixture under conditions appropriate to form the fluoro aryl compound by substituting the leaving group with a F moiety derived from the electrophilic fluorine source. In an exemplary embodiment, the leaving group is a boronic acid moiety or a derivative thereof, e.g., a boronate ester moiety.

According to the method of the invention, any useful temperature or range of temperatures can be used to convert the precursor to the desired product. In various embodiments, the temperature is less than about 300° C., less than about 250° C. or less than about 200° C. In an exemplary embodiment, the reaction mixture is incubated at a temperature from about 20° C. to about 150° C., e.g., about 30° C. to about 100° C., e.g., about 50° C. to about 80° C., e.g., about 50° C. or about 80° C.

The reaction mixture can be incubated for any useful length of time. In various embodiments, the invention is incubated at a desired temperature for about 1 hour to about 36 hours, e.g., for about 6 hours to about 24 hours.

The reaction mixture can be incubated in a vessel of any useful configuration. In an exemplary embodiment, the vessel is sealed while the reaction mixture is incubated, e.g., a sealed tube.

Exemplary embodiments are summarized herein below.

In an exemplary embodiment, the invention provides a composition comprising:

(i) an aryl precursor compound having a leaving group, the compound optionally further substituted at one or more positions;
(ii) an electrophilic fluorine source;
(iii) a metal source; and
(iv) a base.

In an exemplary embodiment, according to the above paragraph, the aryl precursor compound is an aryl boronic acid or a derivative thereof.

In an exemplary embodiment, according to any of the above paragraphs, the aryl precursor compound is an arylboronate ester.

In an exemplary embodiment, according to any of the above paragraphs, the metal source is a copper source.

In an exemplary embodiment, according to any of the above paragraphs, the metal source is (^{t}BuCN)₂CuOTf.

In an exemplary embodiment, according to any of the above paragraphs, the electrophilic fluorine source is a member selected from F-TEDA-BF₄; F-TEDA-PF₆; NFSI; [Cl₂pyF]OTf; [pyF]OTf; [Me₃pyF]BF₄; [Me₃pyF]OTf; and [Me₃pyF]PF₆.

In an exemplary embodiment, according to any of the above paragraphs, the electrophilic fluorine source comprises [Me₃pyF]⁺.

In an exemplary embodiment, according to any of the above paragraphs, the electrophilic fluorine source is [Me₃pyF]PF₆.

In an exemplary embodiment, according to any of the above paragraphs, the base is a fluoride base or an alkoxide base.

In an exemplary embodiment, according to any of the above paragraphs, the base is a member selected from AgF, KF, and CsF.

In an exemplary embodiment, according to any of the above paragraphs, the base is AgF.

In an exemplary embodiment, according to any of the above paragraphs, the composition is anhydrous.

In an exemplary embodiment, according to any of the above paragraphs, the molar ratio of the electrophilic fluorine source to the metal is 1 or greater than 1.

In an exemplary embodiment, according to any of the above paragraphs, the aryl precursor compound, the metal source, the electrophilic fluorine source, and the base are present in the composition in a molar ratio which is about 1:2:3:2.

In an exemplary embodiment, according to any of the above paragraphs, the aryl precursor compound is further substituted with a member selected from ester, ketone, aldehyde, amide, nitrile, halogen, heterocycle and a combination thereof.

In an exemplary embodiment, according to any of the above paragraphs, the aryl precursor compound has the formula:

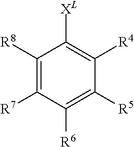

wherein R⁴, R⁵, R⁶, R⁷, and R⁸ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R⁴, R⁵, R⁶, R⁷ and R⁸, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl. R⁹ and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl; and X^{L} is the leaving group.

In an exemplary embodiment, according to any of the above paragraphs, the leaving group is a member selected from:

### —B(OH)₂; —BF₃K;

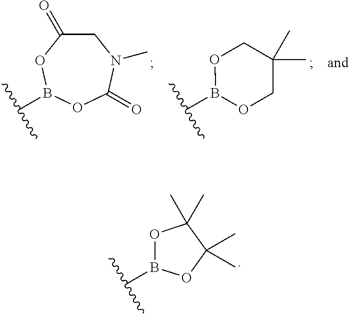

In an exemplary embodiment, according to any of the above paragraphs, the leaving group is:

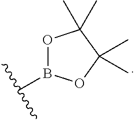 [SMILES: *B1OC(C)(C)C(C)(C)O1]

In an exemplary embodiment, according to any of the above paragraphs, the aryl precursor compound is synthesized in situ.

In an exemplary embodiment, according to any of the above paragraphs, the aryl precursor compound was synthesized in situ from an arene or an aryl bromide.

In an exemplary embodiment, the invention provides a method for forming a fluoroaryl compound, the method comprising: (a) forming a composition according to any of the above paragraphs, wherein the metal source mediates the fluorinating of the aryl precursor compound, at the position of the leaving group, with fluorine derived from the electrophilic fluorine source; and (b) incubating the composition under conditions appropriate to form the fluoroaryl compound.

The following examples illustrate embodiments of the invention and are not intended to limit the scope of the compositions of the invention or the methods in which they find use.

### EXAMPLES

### General Experimental Details

All manipulations were conducted under an inert atmosphere with a nitrogen-filled glovebox unless otherwise noted. All reactions were conducted in oven-dried 4-mL vials fitted with a Teflon-lined screw cap under an atmosphere of nitrogen unless otherwise noted.

Silver fluoride (>99%) was purchased from Acros and used as received. THF was sparged with N₂, passed through activated alumina and stored over 3 Å molecular sieves prior to use. (^{t}BuCN)₂CuOTf was prepared according to our previously published procedure. (Fier, P. S.; Hartwig, J. F. J. Am. Chem. Soc. 2012, 134, 10795.) Unless otherwise noted, all other reagents were purchased from commercial suppliers and used as received.

NMR spectra were acquired on 400 MHz, 500 MHz, or 600 MHz Bruker instruments at the University of California. NMR spectra were processed with MestReNova 5.0 (Mestrelab Research SL). Chemical shifts are reported in ppm and referenced to residual solvent peaks (CHCl₃ in CDCl₃: 7.26 ppm for ¹H and 77.0 ppm for ¹³C) or to an external standard (1% CFCl₃ in CDCl₃: 0 ppm for ¹⁹F). Coupling constants are reported in hertz.

All GC-MS analyses were conducted with an Agilent 6890N GC equipped with an HP-5 column (25 m×0.20 mm ID×0.33 μm film) and an Agilent 5973 Mass Selective Detector. The temperature for each run was held at 50° C. for 2 min, ramped from 50° C. to 300° C. at 40° C./min, and held at 300° C. for 5 min.

### Example 1

### Preparation of 1-fluoro-2,4,6-trimethylpyridinium hexafluorophosphate, [Me₃pyF]PF₆

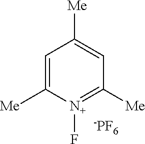

1-fluoro-2,4,6-trimethylpyridinium tetrafluoroborate ([Me₃pyF]BF₄, 4.54 g, 20.0 mmol) was dissolved in water (80 mL), and ammonium hexafluorophosphate (19.56 g, 120 mmol) was added to the resulting solution at once. A white precipitant formed quickly, and the resulting suspension was stirred at room temperature for 2 h. The white solid was collected on a funnel, washed with 3×15 mL of water, 2×15 mL of ether, and dried in vacuo (20 mtorr). 5.00 g (17.5 mmol) of a white powder was obtained, 88% yield.

¹H NMR (400 MHz, CD₃CN) δ 7.65 (d, J=6.4 Hz, 2H), 2.74 (d, J=4.0 Hz, 6H), 2.55 (s, 3H).

¹⁹F NMR (376 MHz, CD₃CN) δ 17.48 (s), −71.40 (d, J=706.4 Hz).

### Example 2

### General Procedure for the Synthesis of Aryl Pinacol Boronate Esters

Into a 20 mL vial was placed the aryl boronic acid (2.0 mmol, 1.0 equiv), pinacol (236 mg, 2.0 mmol, 1.0 equiv), powdered 4 Å molecular sieves (˜300 mg), and 5 mL of ether. The mixture was stirred at room temperature overnight. The molecular sieves were removed by filtration, and the filtrate was concentrated to afford aryl pinacol boronate esters as colorless solids or oils. Further purification of the aryl boronate ester was rarely needed.

### Example 3

### Screen of F⁺ Reagents for the Fluorination of La with (^{t}BuCN)₂CuOTf and AgF

TABLE 1
Screen of F+ Reagents for the Fluorination of 1a with
(^{t}BuCN)₂CuOTf and AgF.

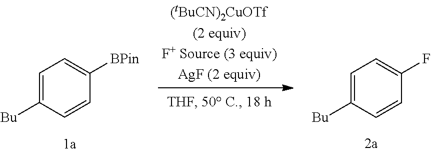

| Entry | F⁺ Source | ArF (%) | ArH (%) | Conversion (%) |
|---|---|---|---|---|
| 1 | F-TEDA-BF₄ | 27 | 37 | 91 |
| 2 | F-TEDA-PF₆ | 26 | 73 | 100 |
| 3 | NFSI | 10 | 90 | 100 |
| 4 | [Cl₂pyF]OTf | 0 | 100 | 100 |
| 5 | [pyF]OTf | 1 | 87 | 100 |
| 6 | [Me₃pyF]BF₄ | 56 | 9 | 84 |
| 7 | [Me₃pyF]OTf | 64 | 13 | 82 |
| 8 | [Me₃pyF]PF₆ | 75 | 12 | 88 |
| 9 | [Me₃pyF]PF₆ | 24 | 57 | 97^{b} |
| 10 | [Me₃pyF]PF₆ | 38 | 39 | 100^{c} |

^{a}Reactions were performed with 0.1 mmol of 1a in 2.0 mL of THF for 18 h. Yields were determined by gas chromatography with 1-bromo-4-fluorobenzene as an internal standard added after the reaction.
^{b}Reactions were performed with KF in place of AgF.
^{c}Reactions were performed with CsF in place of AgF.

Reactions conducted with a series of alkoxide bases gave modest yields (10-15%) of the aryl fluoride product in the presence of (^{t}BuCN)₂CuOTf and [Me₃pyF]PF₆.

### Example 4

### General Procedure for the Fluorination of Aryl Boronate Esters

To an oven-dried 4 mL vial was added AgF (25 mg, 0.2 mmol, 2.0 equiv), (^{t}BuCN)₂CuOTf (76 mg, 0.2 mmol, 2.0 equiv), [Me₃pyF]PF₆ (86 mg, 0.3 mmol, 3.0 equiv) and THF (2.0 mL). The aryl boronate ester (0.1 mmol, 1.0 equiv) was added (solid aryl boronate esters were weighed in the vial prior to adding THF, and liquid aryl boronate esters were added neat by syringe after the addition of THF). The vial was sealed with a Teflon-lined cap and heated at 50° C. with vigorous stirring for 18 h. The solution was allowed to cool to room temperature, and 11.0 μL (0.1 mmol, 1.0 equiv) of 1-bromo-4-fluorobenzene was added as an internal standard. The crude reaction mixture was analyzed by ¹⁹F NMR spectroscopy to determine the yield of aryl fluoride. ¹⁹F NMR chemical shifts were compared to authentic samples of the aryl fluoride product to confirm the identity of the product, and the identities of the products were further assessed by GC/MS.

Fluorination of ArBPin with (^{t}BuCN)₂CuOTf and [Me₃pyF]PF₆

TABLE 2
Fluorination of ArBPin with (^{t}BuCN)₂CuOTf and [Me₃pyF]PF₆^{a}

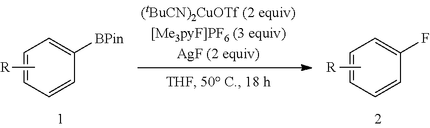

| | |
|---|---|
| 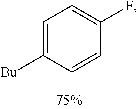 | 2a |
| 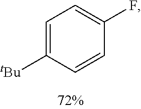 [SMILES: CC(C)(C)c1ccc(F)cc1] | 2b |
| 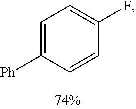 [SMILES: Fc1ccc(-c2ccccc2)cc1] | 2c |
| 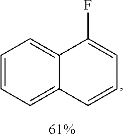 [SMILES: Fc1cccc2ccccc12] | 2d |
| 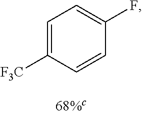 [SMILES: Fc1ccc(C(F)(F)F)cc1] | 2e |
| 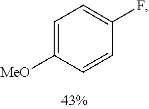 | 2f |
| 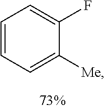 | 2g |
| 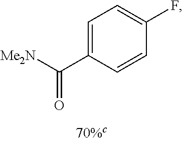 [SMILES: CN(C)C(=O)c1ccc(F)cc1] | 2h |
| 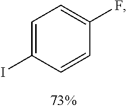 | 2i |
| 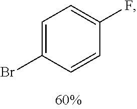 | 2j |
| 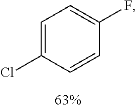 | 2k |
| 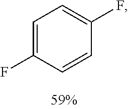 | 2l |
| 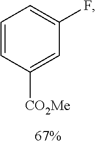 [SMILES: COC(=O)c1cccc(F)c1] | 2m |
| 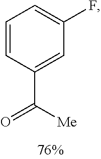 | 2n |
| 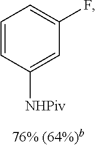 | 2o |
| 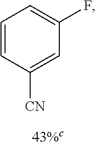 | 2p |
| 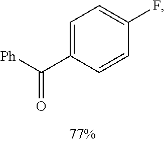 [SMILES: O=C(c1ccccc1)c1ccc(F)cc1] | 2q |
| 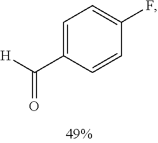 | 2r |
| 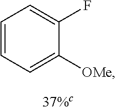 | 2s |
| 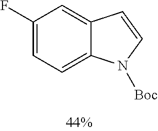 [SMILES: CC(C)(C)OC(=O)n1ccc2cc(F)ccc21] | 2t |
| 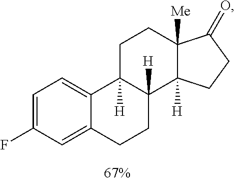 | 2u |
| 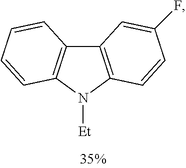 [SMILES: CCn1c2ccccc2c2cc(F)ccc21] | 2v |
| 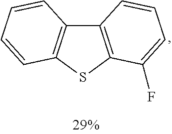 [SMILES: Fc1cccc2c1sc1ccccc12] | 2w |

^{a}Reactions were performed with 0.1 mmol of 1 to determine yields by ¹⁹F NMR spectroscopy with 1-bromo-4-fluorobenzene as an internal standard added after the reaction. ¹⁹F NMR chemical shifts were compared with those of the authentic aryl fluorides.
^{b}Isolated yield from a reaction with 0.5 mmol of ArBPin.
^{c}Reactions were conducted at 80° C.

### Synthesis of N-(3-fluorophenyl)pivalamide (2o)

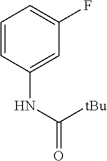 [SMILES: CC(C)(C)C(=O)Nc1cccc(F)c1]

To an oven-dried 20 mL vial was added AgF (127 mg, 1.0 mmol, 2.0 equiv), (^{t}BuCN)₂CuOTf (379 mg, 1.0 mmol, 2.0 equiv), 1-fluoro-2,4,6-trimethylpyridinium hexafluorophosphate (428 mg, 1.5 mmol, 3.0 equiv), to (152 mg, 0.5 mmol, 1.0 equiv) and THF (10 mL). The vial was sealed with a Teflon-lined cap, and the reaction was heated at 50° C. for 18 h. The reaction was cooled, diluted with 15 mL of ether, and filtered through Celite. The filtrate was concentrated and purified by silica gel chromatography eluting with 9:1 hexanes:ethyl acetate (R_{f}=0.18) to afford a white solid (63 mg, 0.32 mmol, 64% yield).

¹H NMR (600 MHz, CDCl₃) δ 7.54 (d, J=11.0 Hz, 1H), 7.35 (s, 1H), 7.25 (t, J=11.4 Hz, 1H), 7.13 (d, J=8.1 Hz, 1H), 6.83-6.77 (m, 1H), 1.31 (s, 9H).

¹³C NMR (151 MHz, CDCl₃) δ 176.60 (s), 163.04 (d, J=244.7 Hz), 139.58 (d, J=11.0 Hz), 129.94 (d, J=9.4 Hz), 115.01 (d, J=2.9 Hz), 110.85 (d, J=21.4 Hz), 107.44 (d, J=26.4 Hz), 39.70 (s), 27.56 (s).

¹⁹F NMR (376 MHz, CDCl₃) δ −111.51-−111.61 (m).

Fluorination of Boronic Acid Derivatives with (^{t}BuCN)₂CuOTf, [Me₃pyF]PF₆ and AgF

TABLE 3
Fluorination of Boronic Acid Derivatives with (^{t}BuCN)₂CuOTf,
[Me₃pyF]PF₆ and AgF.

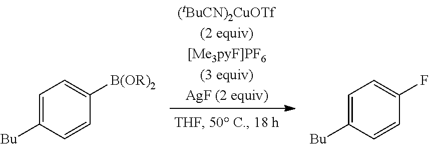

| Entry | (OR)₂ | ArF (%) | ArH (%) |
|---|---|---|---|
| 1 | (OH)₂ | 45 | 5 |
| 2 | BF₃K | 46 | 37 |
| 3 | MIDA | 18 | 23 |
| 4 | Catechol | 0 | 60 |
| 5 | Neopentylglycol | 70 | 15 |
| 6 | Pinacol | 75 | 12 |

^{a}Reactions were performed with 0.1 mmol of aryl-boron in 2.0 mL of THF for 18 h. Yields were determined by gas chroma-tography with 1-bromo-4-fluorobenzene as an internal standard added after the reaction.

### Example 5

### General Procedure for the Fluorination of Arenes Through Ir-Catalyzed C—H Borylation

To an oven-dried 4 mL vial was added arene (0.1 mmol, 1.0 equiv), and 0.2 mL of a stock solution containing 0.1 mol % [Ir(COD)OMe]₂, 0.2 mol % 4,4′-di-tert-butyl bipyridine (dtbpy), and 0.75 equiv of B₂Pin₂. The vial was sealed with a Teflon-lined cap and heated at 80° C. for 18 h. The solution was allowed to cool, and the volatile components were removed in vacuo. To the crude ArBPin was added AgF (25 mg, 0.2 mmol, 2.0 equiv), (^{t}BuCN)₂CuOTf (76 mg, 0.2 mmol, 2.0 equiv), Me₃pyF-PF₆ (86 mg, 0.3 mmol, 3.0 equiv) and THF (2.0 mL). The vial was sealed with a Teflon-lined cap and heated at 50° C. with vigorous stirring for 18 h. The solution was allowed to cool to room temperature, and 11.0 μL (0.1 mmol, 1.0 equiv) of 1-bromo-4-fluorobenzene was added as an internal standard. The crude reaction mixture was analyzed by ¹⁹F NMR spectroscopy to determine the yield of aryl fluoride. ¹⁹F NMR chemical shifts were compared to authentic samples of the aryl fluoride product to confirm the identity of the product, and the identities of the products were further confirmed by GC/MS.

TABLE 4
Fluorination of Arenes via C-H Borylation.

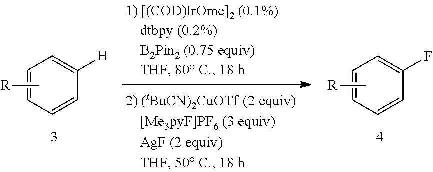

| | |
|---|---|
| 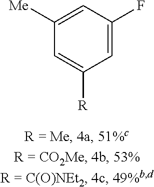 | |
| 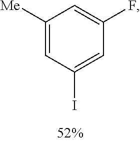 | 4d |
| 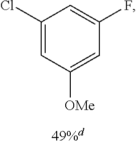 | 4e |
| 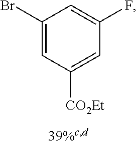 [SMILES: CCOC(=O)c1cc(F)cc(Br)c1] | 4f |

^{a}Reactions were performed with 0.1 mmol of arene. Yields were determined by ¹⁹F NMR spectroscopy with 1-bromo-4-fluorobenzene as an internal standard added after the reaction.
^{b}The borylation reaction was performed with 1.5% [Ir] and 3.0% dtbpy.
^{c}The borylation reaction was performed with 0.5% [Ir] and 1.0% dtbpy.
^{d}The fluorination reaction was performed at 80° C. for 18 h.

### Example 6

### General Procedure for the Fluorination of Aryl Bromides Through Pd-Catalyzed C—Br Borylation

To an oven-dried 4 mL vial was added (dppf)PdCl₂ (2.2 mg, 0.003 mmol, 3 mol %), KOAc (29 mg, 0.3 mmol, 3.0 equiv), B₂Pin₂ (28 mg, 0.11 mmol, 1.1 equiv) and dioxane (0.5 mL). The aryl bromide (0.1 mmol, 1.0 equiv) was added (solid aryl bromides were weighed in the vial prior to adding dioxane, and liquid aryl bromides were added neat by syringe after the addition of dioxane). The vial was sealed with a Teflon-lined cap and heated at 80° C. for 18 h. The solution was allowed to cool and filtered through a short plug of Celite with EtOAc, and the volatile components were removed in vacuo. To the crude ArBPin was added AgF (25 mg, 0.2 mmol, 2.0 equiv), (^{t}BuCN)₂CuOTf (76 mg, 0.2 mmol, 2.0 equiv), [Me₃pyF]PF₆ (86 mg, 0.3 mmol, 3.0 equiv) and THF (2.0 mL). The vial was sealed with a Teflon-lined cap and heated at 50° C. with vigorous stirring for 18 h. The solution was allowed to cool to room temperature, and 11.0 μL (0.1 mmol, 1.0 equiv) of 1-bromo-4-fluorobenzene was added as an internal standard. The crude reaction mixture was analyzed by ¹⁹F NMR spectroscopy to determine the yield of aryl fluoride. ¹⁹F NMR chemical shifts were compared to authentic samples of the aryl fluoride product to confirm the identity of the product. The identities of the products were further confirmed by GC/MS.

TABLE 5
Fluorination of Aryl Bromides via Pd-Catalyzed Borylation.

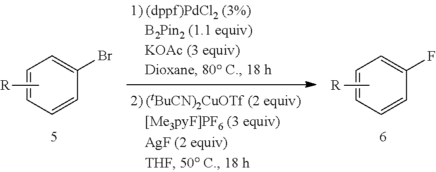

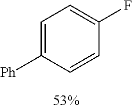 [SMILES: Fc1ccc(-c2ccccc2)cc1]

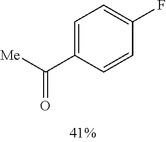

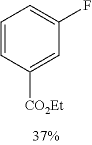 [SMILES: CCOC(=O)c1cccc(F)c1]

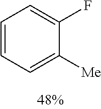

^{a}Reactions were performed with 0.1 mmol of aryl bromide. Yields were determined by ¹⁹F NMR spectroscopy with 1-bromo-4-fluorobenzene as an internal standard added after the reaction.

1. A composition comprising:
(i) an aryl precursor compound having a leaving group, said compound optionally further substituted at one or more positions;
(ii) an electrophilic fluorine source;
(iii) a metal source; and
(iv) a base.

2. The composition according to claim 1, wherein said aryl precursor compound is an aryl boronic acid or a derivative thereof.

3. The composition according to claim 1, wherein said aryl precursor compound is an arylboronate ester.

4. The composition according to claim 1, wherein said metal source is a copper source.

5. The composition according to claim 1, wherein said metal source is (^{t}BuCN)₂CuOTf.

6. The composition according to claim 1, wherein said electrophilic fluorine source is a member selected from F-TEDA-BF₄; F-TEDA-PF₆; NFSI; [Cl₂pyF]OTf; [pyF]OTf; [Me₃pyF]BF₄; [Me₃pyF]OTf; and [Me₃pyF]PF₆.

7. The composition according to claim 1, wherein said electrophilic fluorine source comprises [Me₃pyF]⁺.

8. The composition according to claim 1, wherein said electrophilic fluorine source is [Me₃pyF]PF₆.

9. The composition according to claim 1, wherein said base is a fluoride base or an alkoxide base.

10. The composition according to claim 1, wherein said base is a member selected from AgF, KF, and CsF.

11. The composition according to claim 1, wherein said base is AgF.

12. The composition according to claim 1, wherein said composition is anhydrous.

13. The composition according to claim 1, wherein the molar ratio of said electrophilic fluorine source to the metal is 1 or greater than 1.

14. The composition according to claim 1, wherein said aryl precursor compound, said metal source, said electrophilic fluorine source, and said base are present in said composition in a molar ratio which is about 1:2:3:2.

15. The composition according to claim 1, wherein said aryl precursor compound is further substituted with a member selected from ester, ketone, aldehyde, amide, nitrile, halogen, heterocycle and a combination thereof.

16. The composition according to claim 1, wherein said aryl precursor compound has the formula: 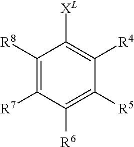
wherein
R⁴, R⁵, R⁶, R⁷, and R⁸ are independently members selected from H, substituted or unsubstituted alkyl, substituted or unsubstituted heteroalkyl, substituted or unsubstituted aryl, substituted or unsubstituted heteroaryl, halogen, CN, CF₃, acyl, —SO₂NR⁹R¹⁰, —NR⁹R¹⁰, —OR⁹, —S(O)₂R⁹, —C(O)R⁹, —COOR⁹, —CONR⁹R¹⁰, —S(O)₂OR⁹, —OC(O)R⁹, —C(O)NR⁹R¹⁰, —NR⁹C(O)R¹⁰, —NR⁹SO₂R¹⁰ and —NO₂, wherein two or more of R⁴, R⁵, R⁶, R⁷ and R⁸, together with the atoms to which they are bonded, are optionally joined to form a ring system which is a member selected from substituted or unsubstituted cycloalkyl, substituted or unsubstituted heterocycloalkyl, substituted or unsubstituted aryl and substituted or unsubstituted heteroaryl,
wherein
R⁹ and R¹⁰ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl, and R⁹ and R¹⁰, together with the atoms to which they are bonded, are optionally joined to form a 5- to 7-membered ring which is a member selected from substituted or unsubstituted cycloalkyl, substituted or unsubstituted heterocycloalkyl, substituted or unsubstituted aryl and substituted or unsubstituted heteroaryl; and
X^{L} is said leaving group.

17. The composition according to claim 1, wherein said leaving group is a member selected from:
hexyleneglycolate; ethyleneglycolate; —B(OH)₂; —BF₃K;

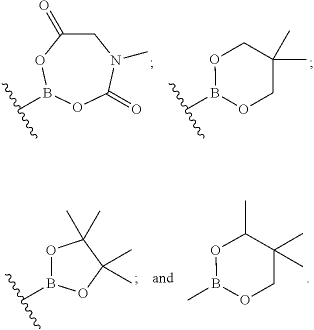

18. The composition according to claim 1, wherein said leaving group is

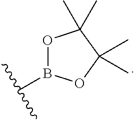 [SMILES: *B1OC(C)(C)C(C)(C)O1]

19. The composition according to claim 1, wherein said aryl precursor compound was synthesized in situ.

20. The composition according to claim 1, wherein said aryl precursor compound was synthesized in situ from an arene or an aryl bromide.

21. A method for forming a fluoroaryl compound, said method comprising:
(a) forming a composition according to claim 1,
wherein the metal source mediates the fluorinating of the aryl precursor compound, at the position of the leaving group, with fluorine derived from the electrophilic fluorine source; and
(b) incubating said composition under conditions appropriate to form said fluoroaryl compound.
